(19) United States
(12) Patent Application Publication
(10) Pub. No.: US 2011/0165560 A1
(43) Pub. Date: Jul. 7, 2011

(54) METHODS AND COMPOSITIONS FOR SCREENING FOR INDIVIDUALS AT RISK FOR SUCCINATE DEHYDROGENASE-RELATED DISEASE CONDITIONS

(76) Inventors: JARED RUTTER, SALT LAKE CITY, UT (US); HUAIXIANG HAO, MALDEN, MA (US); JOSHUA SCHIFFMAN, SALT LAKE CITY, UT (US)

(21) Appl. No.: 12/831,141

(22) Filed: Jul. 6, 2010

Related U.S. Application Data
(60) Provisional application No. 61/270,259, filed on Jul. 6, 2009.

(51) Int. Cl. C12Q 1/68; G01N 33/48; C12N 15/63

(52) U.S. Cl. 435/6.1; 436/94; 435/320.1

(57) ABSTRACT

The present invention provides methods, compositions, and kits associated with succinate dehydrogenase-related disease conditions. In one aspect of the present invention, a method for screening a test subject to determine risk for developing a succinate dehydrogenase-related disease condition is provided. Such a method can include obtaining a biological sample from the test subject and identifying a mutation in gene hSDH5 from the biological sample of the test subject, wherein the mutation effectuates a decreased level of succinate dehydrogenase flavination in the test subject as compared to a level of succinate dehydrogenase flavination in a normal subject. In one aspect, the mutation is at hSDH5 Gly78 of gene hSDH5. In another aspect, the mutation is equivalent to an hSDH5 Gly78Arg substitution of gene hSDH5.

### PRIORITY DATA

This application claims the benefit of U.S. Provisional Patent Application Ser. No. 61/270,259, filed on Jul. 6, 2009, which is incorporated herein by reference in its entirety.

### GOVERNMENT INTEREST

This invention was made with government support under NIH grants DK071962 and GM087346. The United States government has certain rights to this invention.

### BACKGROUND OF THE INVENTION

Mitochondria are complex organelles that play various roles in many aspects of cellular function, including ATP production, intermediary metabolism, apoptosis, and many others. As a result, mitochondrial dysfunction is tightly coupled with a variety of human diseases, including cancer, type 2 diabetes, and various age-related pathologies. The link between mitochondrial metabolism and cancer is a particularly emergent area of interest and investigation. Cancer-related mitochondrial defects have been identified, such as altered expression and activity of respiratory chain and tricarboxylic acid cycle enzymes and mitochondrial DNA (mtDNA) mutations. Specific discussion can be found in Baysal et al., (2000), Mutations in SDHD, a mitochondrial complex II gene, in hereditary paraganglioma, Science 287, 848-851; Chatterjee et al., (2006), Mitochondrial DNA mutations in human cancer, Oncogene 25, 4663-4674; and Yan et al., (2009), IDH1 and IDH2 mutations in gliomas, N Engl J Med 360, 765-773, each of which are incorporated herein by reference. The discovery of the central role of mitochondria in apoptosis further emphasizes the importance of mitochondria in cancer, which typically exhibits altered sensitivity to apoptotic cell death.

### BRIEF DESCRIPTION OF THE DRAWINGS

FIG. 1 shows sequence alignments of Sdh5 orthologs according to one aspect of the present invention.

FIGS. 2A-C provides results showing that the sdh5Δ mutant is respiratory deficient but has intact mitochondrial DNA according to another aspect of the present invention.

FIGS. 3A-D provides results showing that Sdh5 is a soluble mitochondrial matrix protein according to another aspect of the present invention.

FIG. 4 provides results showing that Sdh5 deletion causes hydrogen peroxide hypersensitivity according to another aspect of the present invention.

FIG. 5 provides results showing that Sdh5 deletion leads to decreased chronological life span according to another aspect of the present invention.

FIGS. 6A-B provides results showing that Sdh5 physically interacts with Sdh1 according to another aspect of the present invention.

FIG. 7 provides results showing weak growth of sdhΔ strains with ethanol as the carbon source according to another aspect of the present invention.

FIGS. 8A-F provides results showing that Sdh5 is required for succinate dehydrogenase complex activity and stability according to another aspect of the present invention.

FIGS. 9A-D provides results showing that Sdh5 is necessary and sufficient for flavination of Sdh1 according to another aspect of the present invention.

FIG. 10 shows a graphical representation of the G78R mutation in PGL2 patients according to another aspect of the present invention.

FIGS. 11A-D provides results showing that human SDH5 is mutated in a Dutch lineage with paraganglioma according to another aspect of the present invention.

FIGS. 12A-D provides results showing that hSDH5 G78R is a loss of function mutation according to another aspect of the present invention.

FIG. 13 provides results showing that overexpression of SDH complex subunits and chaperone does not rescue the glycerol growth defect of the sdh5Δ mutant strain according to another aspect of the present invention.

FIG. 14 provides results showing that overexpression of Sdh5 fails to rescue the glycerol growth defect of flx1Δ mutant strain according to another aspect of the present invention.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a promoter” includes one or more of such promoters and reference to “the protein” includes reference to one or more of such proteins.

Definitions

In describing and claiming the present invention, the following terminology will be used in accordance with the definitions set forth below.

As used herein, “subject” refers to an organism that includes SDH proteins.

As used herein, the term “biological sample” refers to any material of a biological nature obtained from a subject from which DNA can be obtained. Non-limiting examples include biological fluids and biological tissues such as blood, blood serum, saliva, semen, vaginal fluid, lymph, urine, lachrymal fluid, cancerous tissue, non-cancerous tissue, tumor tissue, skin tissue, and the like.

As used herein, the term “succinate dehydrogenase-related disease condition” refers to a disease condition that is linked in some way to defective functionality of succinate dehdrogenase. As an example, a succinate dehydrogenase-related cancer would be any cancer that is linked to defective functionality of succinate dehydrogenase.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Invention

The present invention provides methods, compositions, and kits associated with succinate dehydrogenase-related disease conditions. In one aspect of the present invention, a method for screening a test subject to determine risk for developing a succinate dehydrogenase-related disease condition is provided. Such a method can include obtaining a biological sample from the test subject and identifying a mutation in gene hSDH5 from the biological sample of the test subject, wherein the mutation effectuates a decreased level of succinate dehydrogenase flavination in the test subject as compared to a level of succinate dehydrogenase flavination in a normal subject. It should be noted that one of ordinary skill in the art is capable of readily establishing the level of succinate dehdryogenase flavination in a normal subject. Additionally, obtaining biological samples and testing those samples for gene mutations are well known in the art, and one of ordinary skill in the art would be readily able to test for mutations of the present invention once in possession of the present disclosure.

In one aspect, the mutation is at hSDH5 Gly78 of gene hSDH5. In another aspect, the mutation is equivalent to an hSDH5 Gly78Arg substitution of gene hSDH5. Additionally, non-limiting examples of biological samples includes biological fluids, biopsies, tumors, cancerous tissue, noncancerous tissue, and combinations thereof. In another aspect, the decreased level of succinate dehydrogenase flavination is a substantially complete or complete absence of succinate dehydrogenase flavination. In yet another aspect, the decreased level of succinate dehydrogenase flavination is at least a 50% decrease as compared to a normal subject. In a further aspect, the decreased level of succinate dehydrogenase flavination is at least a 75% decrease as compared to a normal subject.

A variety of succinate dehydrogenase-related disease conditions are implicated and within the scope of the present invention. In one aspect, for example, the disease condition is a succinate dehydrogenase-related cancer. Other non-limiting examples of such disease conditions can include neuroendocrine tumors, paraganglioma tumors, gastrointestinal tumors, Carney-Stratakis syndrome, phenochromocytoma tumors, renal cell carcinomas, optic atrophy, ataxia, myopathies, neurodegeneration, and combinations thereof. In one specific aspect, the disease condition is a paraganglioma tumor.

The results of identifying a mutation in gene hSDH5 can be utilized for various purposes. Non-limiting examples can include predicting the disease condition risk, predicting the disease condition progression, predicting genetic inheritance risks associated with the disease condition, making a clinical diagnosis of the disease condition, providing information to affect the course of the disease condition, adjusting clinical therapy to treat the disease condition, and the like, including combinations thereof.

The present invention also provides compositions containing nucleotide constructs of hSDH5 mutants. In one aspect, such a composition can include a nucleotide construct of a mutant of hSDH5 and a member selected from the group consisting of a vector, RNA, a virus, and combinations thereof. In one aspect, the composition is a vector including the nucleotide construct of mutant hSDH5. In another aspect, the mutant hSDH5 encodes a Gly78 mutation. In yet another aspect, the mutant hSDH5 encodes a Gly78Arg substitution.

The present invention also provides kits for screening a test subject. In one aspect, for example, a kit for screening a test subject to determine whether the subject is at risk for developing a succinate dehydrogenase-related disease condition can include a kit housing containing an assay capable of identifying a mutation in gene hSDH5 from the biological sample of the test subject, and instructions describing how to use the assay to screen the test subject for the disease condition associated with succinate dehydrogenase flavination. In one specific aspect, the assay identifies the mutation as being at hSDH5 Gly78 of gene hSDH5. It should be understood that one of ordinary skill in the art would readily understand what components and ingredients to include in such a kit once in possession of the present disclosure.

In another aspect, for example, a method for screening a test subject to determine risk for developing a succinate dehydrogenase-related disease condition is provided, including identifying a decreased level of succinate dehydrogenase flavination in the test subject as compared to a level of succinate dehydrogenase flavination in a normal subject. In another aspect, the decreased level of succinate dehydrogenase flavination is a substantially complete or complete absence of succinate dehydrogenase flavination. In one specific aspect, identifying the decreased level of succinate dehydrogenase flavination includes detecting a decrease in SDH1-FAD conjugates in biological sample from the test subject as compared to a level of SDH1-FAD conjugates in a normal subject. In another specific aspect, the decrease in SDH1-FAD conjugates in biological sample from the test subject includes a substantially complete or complete absence of SDH1-FAD conjugates.

It is contemplated that various factors can contribute to a decreased level of succinate dehydrogenase flavination. In one aspect, for example, the decreased level of succinate dehydrogenase flavination is characterized by a mutation in gene SDH5. In another aspect, the mutation is at hSDH5 Gly78 of gene SDH5. In yet another aspect, the mutation is equivalent to hSDH5 Gly78Arg in gene SDH5.

Succinate dehydrogenase (SDH) is an enzyme complex that is bound to the inner mitochondrial membrane in eukaryotic organisms and in some prokaryotes. SDH is known to participate in both the electron transport chain and the citric acid cycle. In the citric acid cycle, SDH catalyzes the oxidation of succinate to fumarate with the reduction of ubiquinone to ubiquinol as part of the energy production processes of the mitochondria. The SDH complex includes at least 4 major subunits, SDHA-D. It should be noted that subunits SDHA-D are synonymously known as Sdh1-4 (orthologs in yeast), and as such, these terms can be used interchangeably. SDH also contains multiple cofactors that play roles in the function of the complex. For example, an iron-sulfur cluster is present in SDHB and a heme is shared by SDHC and SDHD. Subunit SDHA includes flavin adenine dinucleotide (FAD) inserted covalently into SDHA in a process known as flavination, without which the succinate to fumarate oxidation does not occur.

A project was initiated to understand the function of a subset of previously uncharacterized mitochondrial proteins. Yeast was utilized as the primary model system for at least three reasons. First, the yeast S. cerevisiae is a tractable and facile genetic and biochemical system. Second, mitochondrial biology is cell-autonomous and highly conserved from yeast to humans. Finally, yeast can survive the complete loss of respiration, due to their ability to generate ATP from fermentation. This is important for genetic analysis of proteins that are essential for respiration, the deletion of which would be lethal in other organisms. This project focused on the protein EMI5/YOL071W. EMI5 (Early Meiotic Induction 5) was so named because it was initially identified in screens for sporulation (meiosis) mutants, but no additional work has been described. Mitochondrial proteins may affect sporulation due to the necessity of respiration in this process. Through this project, a novel protein interaction between the previously uncharacterized EMI5 protein and the SDH complex was discovered that may allow for the identification of subjects at risk for SDH-related disease conditions, as well as the potential diagnosis, prognosis, and treatment of such diseases.

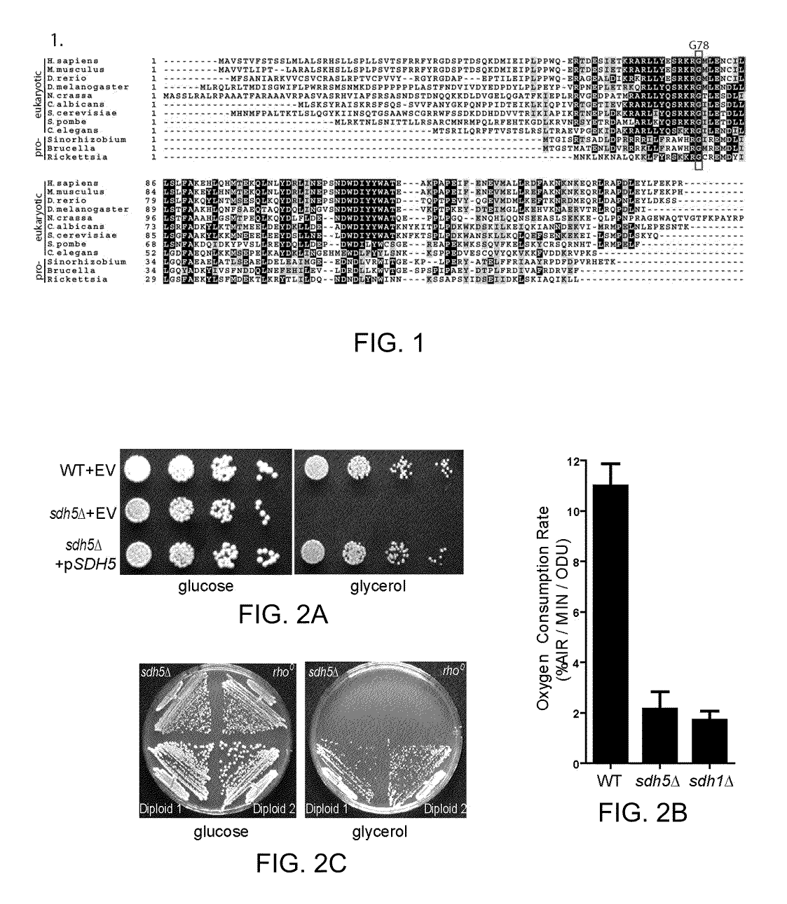

Based on the molecular function that was discovered, EMI5 was renamed to Sdh5. As is shown in FIG. 1, the Sdh5 protein family is highly conserved throughout eukarya and in some prokaryotic species. Sdh5 sequences from nine diverse eukaryotic species (top 9) and three prokaryotic species (bottom 3) are shown (SEQ IDs 01-12). The G78 residue found to be mutated in PGL2 patients is indicated (see below). Despite the high degree of conservation, there appear to be no known functional domains predicted and no known studies of any orthologs have been described. It was discovered that Sdh5 is a soluble mitochondrial matrix protein that is required for respiration, and is required for the activity and stable assembly of the SDH complex. Tandem affinity purification followed by mass spectrometry revealed that Sdh5 interacts with the Sdh1 catalytic subunit (SDHA) of the SDH complex. Sdh5, however, appears to not be a stable part of the SDH complex, but rather is necessary and sufficient for FAD conjugation (flavination) of Sdh1. Loss of Sdh5 leads to a loss of SDH activity, probably a result of the fact that Sdh5 is necessary and sufficient for insertion of the obligate FAD cofactor in Sdh1.

The human Sdh5 ortholog similarly interacts with human SDHA (Sdh1 ortholog) and is able to complement the yeast sdh5Δ mutant phenotype and rescue Sdh1 flavination, suggesting functional conservation. SDH deficiency in humans has been linked to paraganglioma (PGL), neuroendocrine tumors derived from the extra-adrenal paraganglia of the autonomic nervous system. Familial PGL syndromes have been mapped to four loci, designated PGL1-4. Mutations in three of the SDH structural subunits, SDHB, SDHC and SDHD, have been associated with PGL4, PGL3 and PGL1, respectively. The PGL2 gene was mapped to an interval on chromosome 11 in a Dutch lineage, but the affected gene eluded identification. That interval includes human Sdh5, and the inventors have now identified an hSdh5 G78R mutation that segregates with disease in PGL2 patients and causes loss of SDHA flavination. Using cultured human cells and taking advantage of hSdh5 complementation of the yeast sdh5Δ mutant phenotype, it is now shown that this mutation causes a loss of function. Additionally, the G78R mutant of hSdh5 was inactive in functional studies and tumors derived from PGL2 patients exhibited a near complete loss of SDHA flavination. Thus, starting with an uncharacterized yeast protein, the inventors have determined Sdh5's molecular function and the causal role of its human ortholog in a familial neuroendocrine tumor syndrome. Further discussion of PGL1-4 can be found in Favier et al., (2005), Hereditary paraganglioma/pheochromocytoma and inherited succinate dehydrogenase deficiency, Horm Res 63, 171-179; and in Mariman et al., (1993), Analysis of a second family with hereditary non-chromaffin paragangliomas locates the underlying gene at the proximal region of chromosome 11q, Hum Genet 91, 357-361; both of which are incorporated herein by reference.

Thus, in one aspect of the present invention a method for screening a test subject to determine risk for developing a succinate dehydrogenase-related disease condition is provided. Such a method can include obtaining a biological sample from the test subject, and identifying a mutation in gene hSDH5 from the biological sample. It should be noted that gene hSDH5 may also be referred to as gene SDHAF2. In this case, the mutation effectuates a decreased level of succinate dehydrogenase flavination in the test subject as compared to a level of succinate dehydrogenase flavination in a normal subject. Thus subjects can be screened for mutations in hSDH5 as part of any genetic testing regime. This can be performed in subjects due to a suspected risk, as would be the case for subjects from families known to carry the mutant gene, or it can be performed as part of standard genetic testing.

Positive results indicating the presence of the mutant gene can be utilized for a variety of uses. For example, a subject testing positive for the mutant gene can be more closely monitored so that treatment can begin quickly once a disease condition is initiated. In many cases, particularly those involving cancers, frequent testing can identify a disease condition long before it becomes apparent to the subject. Such early monitoring and detection can greatly improve the prognosis of the disease condition, and in many cases increase the likelihood of survival for the affected subjects. In some cases, pretreatment for the disease condition can also be initiated. Other uses can include, without limitation, predicting the progression of a disease condition, predicting genetic inheritance risks associated with the disease condition, making a clinical diagnosis of the disease condition, providing information to affect the course of the disease condition, adjusting clinical therapy to treat the disease condition, and the like.

Sdh5 is a Soluble Mitochondrial Matrix Protein

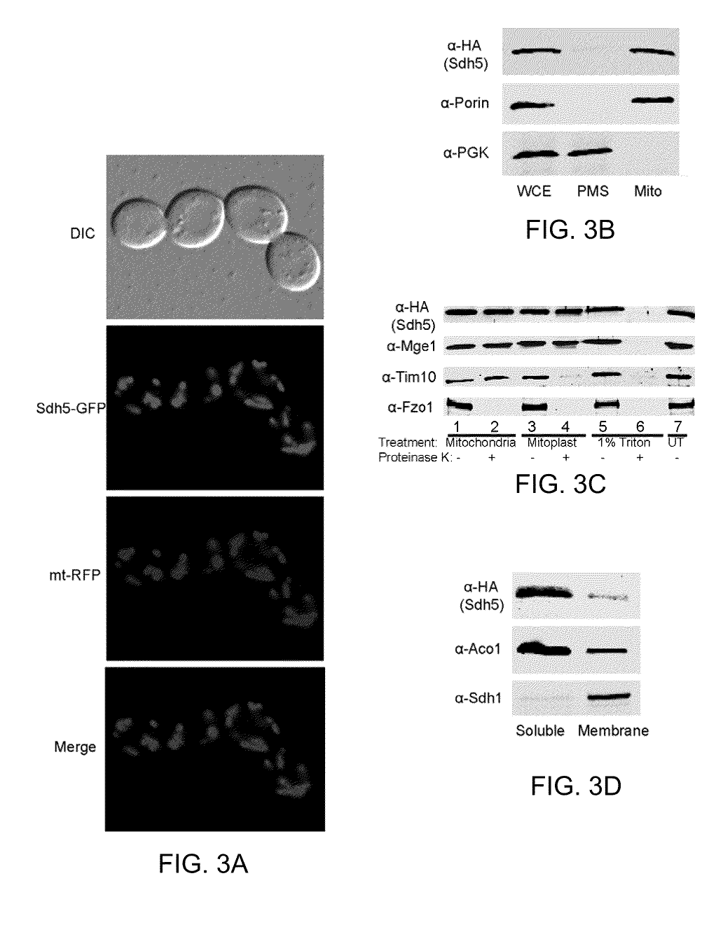

To confirm the mitochondrial localization of Sdh5 by fluorescence microcopy and subcellular fractionation, strains of yeast were generated wherein deletion of the endogenous SDH5 gene (sdh5Δ, JRY609) was complemented by plasmids that express C-terminally GFP-tagged or HA-tagged forms of Sdh5 under the control of the native SDH5 promoter (pSdh5-GFP, pSdh5-HA). Both forms of tagged Sdh5 are functional, as they complement the sdh5Δ mutant phenotype (FIG. 2A and data not shown). Sdh5-GFP colocalized with the red fluorescent mitochondrial marker mito-RFP, indicating its constitutive mitochondrial localization (FIG. 3A). This was the case in glucose, raffinose, or glycerol medium (data not shown). When cell lysates were fractionated by differential centrifugation, Sdh5-HA was exclusively detected in mitochondria, as was the mitochondrial marker Porin (FIG. 3B). In contrast, Sdh5-HA was not found in the post-mitochondrial supernatant, which contains the cytoplasmic protein 3-phosphoglycerate kinase (PGK).

The sub-mitochondrial localization of Sdh5 was examined by proteinase K (PK) protection (FIG. 3C). When intact mitochondria were treated with PK, the outer membrane protein Fzo1 was efficiently degraded, but the intermembrane space protein Tim10 was protected, demonstrating the integrity of the outer membrane. Tim10 was degraded by PK after hypotonic swelling of mitochondria, due to rupture of the outer membrane (generating mitoplasts), while the matrix protein Mge1 was still protected by the undisrupted inner membrane. After both membranes were solubilized by treatment with 1% Triton X-100, all proteins including Sdh5-HA were efficiently degraded by PK. The degradation pattern of Sdh5-HA is the same as that of the matrix protein Mge1, demonstrating that Sdh5 localizes to the mitochondrial matrix.

To assess whether Sdh5 is a soluble matrix protein or associates with the inner membrane, isolated mitochondria were sonicated in detergent-free buffer and separated into soluble and insoluble membrane fractions by ultracentrifugation. The majority of Sdh5-HA and aconitase (a soluble matrix protein) were found in the soluble fraction, while the peripheral membrane protein Sdh1 was found predominantly in the membrane fraction. Together, these data show that Sdh5 is a soluble mitochondrial matrix protein.

Sdh5 is Required for Respiration

One function of mitochondria across phylogeny is to generate ATP for cellular activities and growth through aerobic respiration. Yeast have the ability to generate ATP and grow in the absence of respiration via fermentative metabolism. As a result, respiratory-deficient mutants are viable on fermentable carbon sources such as glucose, but are inviable on non-fermentable carbon sources such as glycerol. To investigate whether Sdh5 is required for mitochondrial respiration, the growth of wild-type (WT) and sdh5Δ mutant yeast were compared on glucose and glycerol medium (FIG. 2A). While deletion of Sdh5 had no effect on growth on glucose medium, the sdh5Δ strain exhibited a complete loss of growth on glycerol medium. This glycerol growth defect of the sdh5Δ mutant was rescued by the pSdh5-HA plasmid (FIG. 2A) and the pSdh5-GFP plasmid (data not shown), demonstrating that loss of Sdh5 is responsible for the growth defect, and that these two plasmids express functional Sdh5.

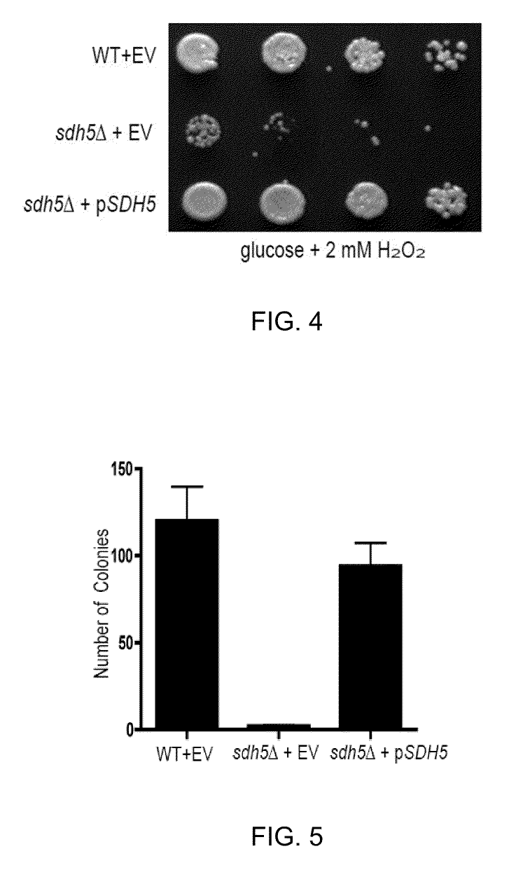

It was also discovered that the sdh5Δ mutant strain had the respiration-related phenotypes of increased sensitivity to hydrogen peroxide and decreased chronological lifespan (FIGS. 4 and 5). Regarding FIG. 4, serial 5-fold dilutions of saturated SD-Ura liquid culture of each indicated strain were spotted on SD-Ura solid medium containing 2% glucose and 2 mM H2O2 and grown at 30° C. for 2 days. Regarding FIG. 5, each indicated strain was cultured in SD-Ura liquid medium at 30° C. for 7 days. Dilutions from each culture containing 200 cells calculated from OD were plated onto SD-Ura solid medium and grown at 30° C. for 2 days. The number of colonies on each plate was counted and data represent the average±standard deviation of 3 replicates per strain.

To directly assess respiration, the rate of oxygen consumption was measured and found to be decreased more than five-fold in the sdh5Δ mutant compared to wild-type (FIG. 2B). The residual oxygen consumption in the sdh5Δ mutant is similar to an sdh1Δ strain, which lacks SDH Complex II of the electron transport chain and is generally accepted as being respiratory deficient (See Chapman et al., (1992), Sdh1, the gene encoding the succinate dehydrogenase flavoprotein subunit from Saccharomyces cerevisiae, Gene 118, 131-136, incorporated herein by reference.) Combining the glycerol growth defect and decreased oxygen consumption of the sdh5Δ mutant, it can be concluded that Sdh5 is required for mitochondrial respiration.

Mutation or loss of the mitochondrial genome (mtDNA) is one common cause of respiratory deficiency in yeast. This explanation is unlikely in the present case because the sdh5Δ mutant phenotype is rescued by subsequent ectopic expression of Sdh5, and as such, this rescue is inconsistent with irreversible mtDNA mutation or loss. However, the integrity and function of mtDNA in sdh5Δ haploids was specifically tested by mating with rho⁰ haploids (FIG. 2C). A rho⁰ strain has lost mtDNA and therefore cannot grow on glycerol medium. Two independent diploids from the mating of sdh5Δ and rho⁰ haploids both grow normally on glycerol medium, however. This indicates that the mtDNA of the sdh5Δ haploid is functional, as it is able to complement the lack of mtDNA from the rho⁰ haploid. Therefore, the sdh5Δ mutant strain exhibits complete respiratory deficiency, in spite of having an intact and functional mitochondrial genome.

Sdh5 Interacts with Sdh1

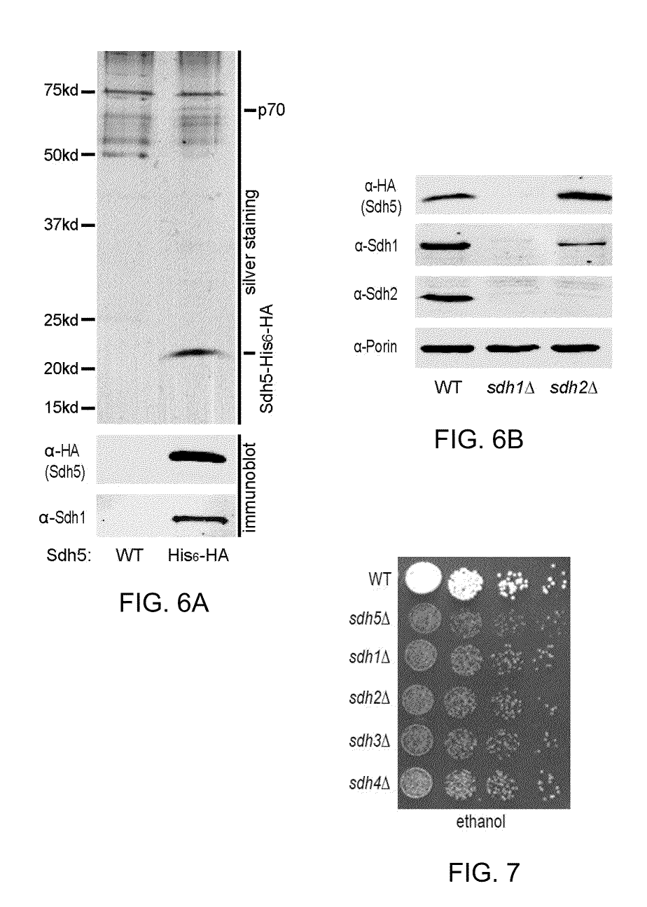

To understand the role of Sdh5 in respiration, physical interactor(s) of Sdh5 by tandem affinity purification (TAP) were identified. An SDH5 allele was generated expressing a C-terminal polyhistidine and dual HA tagged Sdh5 fusion from the native SDH5 promoter (JRY597). This strategy has been shown to enable a similar degree of purification to the classical CBP/ProteinA TAP tag (See Honey et al., (2001), A novel multiple affinity purification tag and its use in identification of proteins associated with a cyclin-CDK complex, Nucleic Acids Res 29, E24, incorporated herein by reference). The Sdh5-His-HA fusion protein was purified from 50mg of isolated mitochondria, solubilized with 0.1% NP-40 detergent, using standard nickel chromatography followed by anti-HA affinity chromatography, eluting with HA peptide. Silver staining of the final elution samples showed a number of bands that were present in both the Sdh5-His-HA and untagged control samples. The 22 kD tagged Sdh5, the identity of which was confirmed by anti-HA immunoblot, is shown in FIG. 6A. A 70 kD band that was present only in the Sdh5-His-HA sample but not the control can also be seen. This protein was identified by mass spectrometry as Sdh1, the catalytic subunit of the succinate dehydrogenase (SDH) complex. The specific presence of Sdh1 in the final elution was confirmed by immunoblot with anti-Sdh1 antibody (FIG. 6A).

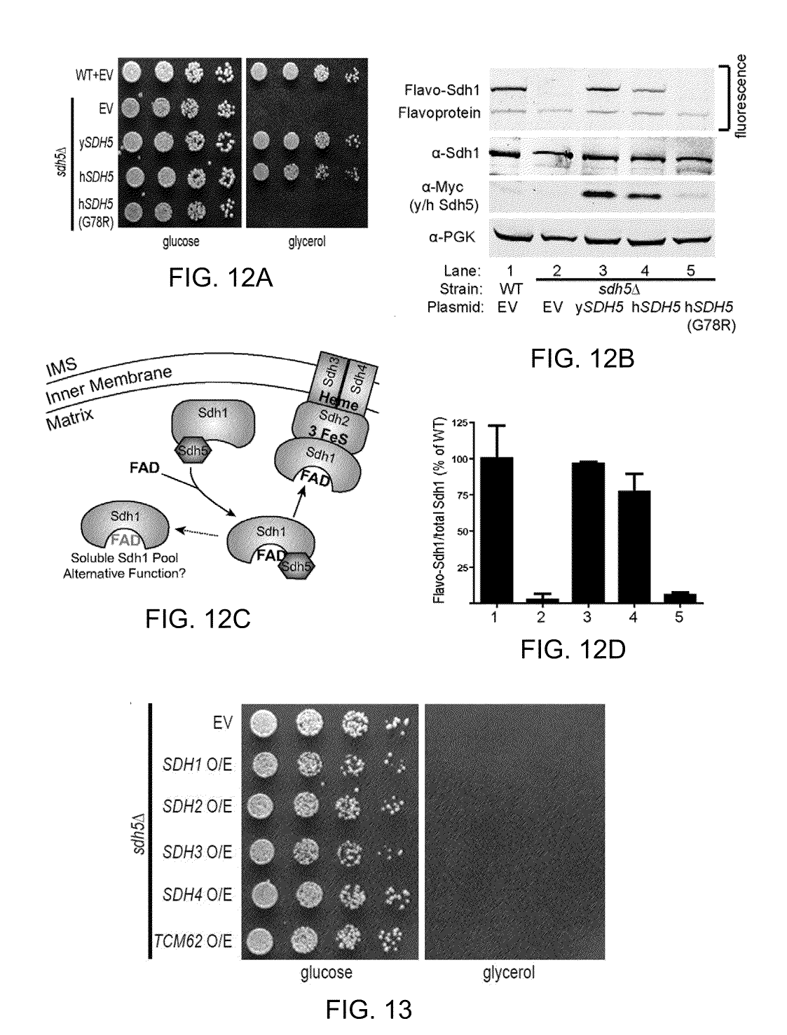

The SDH complex plays a role in both the TCA cycle and the electron transport chain (ETC), where it is often referred to as Complex II. The SDH complex oxidizes succinate to fumarate, coupled with electron transfer to Coenzyme Q and along the ETC eventually to oxygen. During this process a proton gradient is established across the inner membrane, which drives the synthesis of ATP through Complex V. The SDH complex is a heterotetramer and is highly conserved throughout eukaryotes (Sdh1-4 in yeast and SDHA-D in mammals). As illustrated in FIG. 12C, subunits 1 and 2 form the catalytic dimer and subunits 3 and 4 form a membrane-associated structural dimer, which anchors the catalytic dimer on the matrix side of the inner membrane.

Sdh1 was an intriguing potential binding protein due to the similarity between the sdh1Δ and sdh5Δ mutant phenotypes. Specifically, both phenotypes are unable to grow on glycerol medium, but are able to grow weakly with ethanol as the carbon source (FIG. 7). Regarding FIG. 7, serial 5-fold dilutions of saturated YPAD liquid culture of each indicated strain were spotted on YPA solid medium with 3% ethanol as the carbon source and grown at 30° C. for 3 days. Further, these mutant strains were previously found to share an acetate hyper-excretion phenotype, found in only four other mutants, all lacking components of the TCA cycle. The importance of the Sdh1-Sdh5 interaction was confirmed by the observation that Sdh5 fails to accumulate in the sdh1Δ mutant mitochondria (FIG. 6B), presumably due to degradation of Sdh5 in the absence of the Sdh1/Sdh5 complex. Specifically, no Sdh2 protein was detected in sdh1Δ mutant mitochondria and the Sdh1 level was significantly reduced (but not absent) in sdh2Δ mitochondria. In contrast to the destabilization of Sdh5 in the sdh1Δ mutant, loss of SDH2 leads to a more than 2-fold increase in the Sdh5 protein level (FIG. 6B), presumably due to increased Sdh1/Sdh5 complex formation in the absence Sdh2, the major Sdh1 binding partner.

Sdh5 is Required for SDH Activity

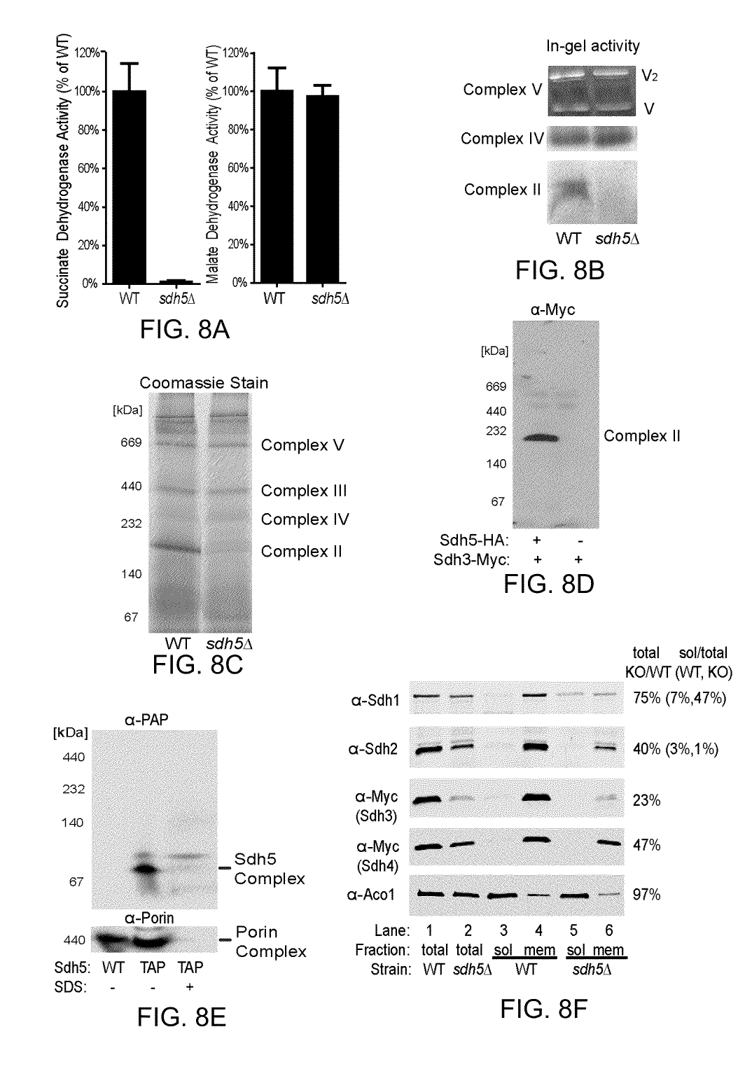

To examine the functional significance of the Sdh1-Sdh5 interaction, SDH activity in sdh5Δ mutant mitochondria was measured. There was no detectable SDH activity in the sdh5Δ mutant (FIG. 8A), as has been observed for an sdh1Δ mutant. The activity of malate dehydrogenase, another enzyme in the TCA cycle, was not affected by SDH5 deletion (FIG. 8A). Because SDH also functions as Complex II in the ETC, in-gel activity staining after separation of mitochondrial membrane complexes by blue native-polyacrylamide gel electrophoresis (BN-PAGE) was also performed. As shown in FIG. 8B, Complex II activity is absent in the sdh5Δ mutant, while the activity of Complexes IV and V are normal. S. cerevisiae lacks Complex I, and there is no BN-PAGE compatible in-gel assay for complex III activity. Therefore, it is concluded that the sdh5Δ mutant strain exhibits a specific loss of SDH activity.

SDH is a complex assembled from four distinct nuclear-encoded subunits. It may be hypothesized that Sdh5 might be required for the synthesis or assembly of Complex II. To test this hypothesis, respiratory complex formation was examined by Coomassie blue staining after BN-PAGE, and it was discovered that the band corresponding to Complex II in wild-type mitochondria is absent in sdh5Δ mutant mitochondria (FIG. 8C). The other complexes appear normal in the sdh5Δ mutant (FIG. 8C). Loss of Complex II in the sdh5Δ mutant was confirmed by anti-Myc immunoblot after BN-PAGE of wild-type and sdh5Δ strains expressing Myc-tagged Sdh3 (FIG. 8D).

One explanation for the absence of Complex II in sdh5Δ mutant mitochondria is that Sdh5 is a necessary component of the SDH complex that has somehow eluded detection previously. To address this possibility, the Sdh5-containing complex was visualized by BN-PAGE followed by immunoblot as described above. As shown in FIG. 8E, the Sdh5-TAP fusion protein exists as an approximately 90 kD complex, which is larger than an Sdh5-TAP monomer (37 kD), but is too small to be Complex II (around 200 kD). This Sdh5 complex is specific, as it was disrupted by the addition of the denaturant SDS before electrophoresis. This 90 kD complex is likely to be the Sdh5-TAP/Sdh1 (70 kD) heterodimer observed in FIG. 6A.

As Complex II is either not assembled or is unstable in sdh5Δ mutant mitochondria, the steady-state levels of each subunit were determined. All four SDH subunits were significantly decreased in the sdh5Δ mutant (FIG. 8F, lane 1 versus lane 2). This decrease in protein levels may be due to degradation in the absence of stable Complex II assembly. The abundance of a distinct mitochondrial matrix protein, aconitase, was similar between a wild-type and sdh5Δ mutant strain. Sdh1 and Sdh2 membrane association was also examined as an alternative way to assess Complex II assembly in sdh5Δ mutant mitochondria in addition to the previously described BN-PAGE technique. In wild-type mitochondria, both Sdh1 and Sdh2 exist predominantly in the membrane fraction (93% and 97%, respectively) by virtue of complex formation with the Sdh3 and Sdh4 membrane subunits (FIG. 8F, lanes 3 and 4). In sdh5Δ mutant mitochondria, Sdh2 as well as Sdh3 and Sdh4 were still observed exclusively in the membrane fraction, albeit at a significantly reduced level (FIG. 8F, lanes 5 and 6). Sdh1, however, was present about equally in the membrane and soluble fractions (53% and 47%, respectively). This indicates that Sdh1 interacts more weakly with Complex II in sdh5Δ mutant mitochondria and that Sdh1 is much more stable as a soluble matrix protein than the other Complex II subunits including Sdh2. Taking these data together, it appears that Complex II assembles in the absence of Sdh5, but that the assembled complex is non-functional and Sdh1 is not incorporated stably. As a result, the unstable complex is more susceptible to degradation and is disrupted by detergent extraction during the BN-PAGE procedure (fractionation in FIG. 8F is detergent-free).

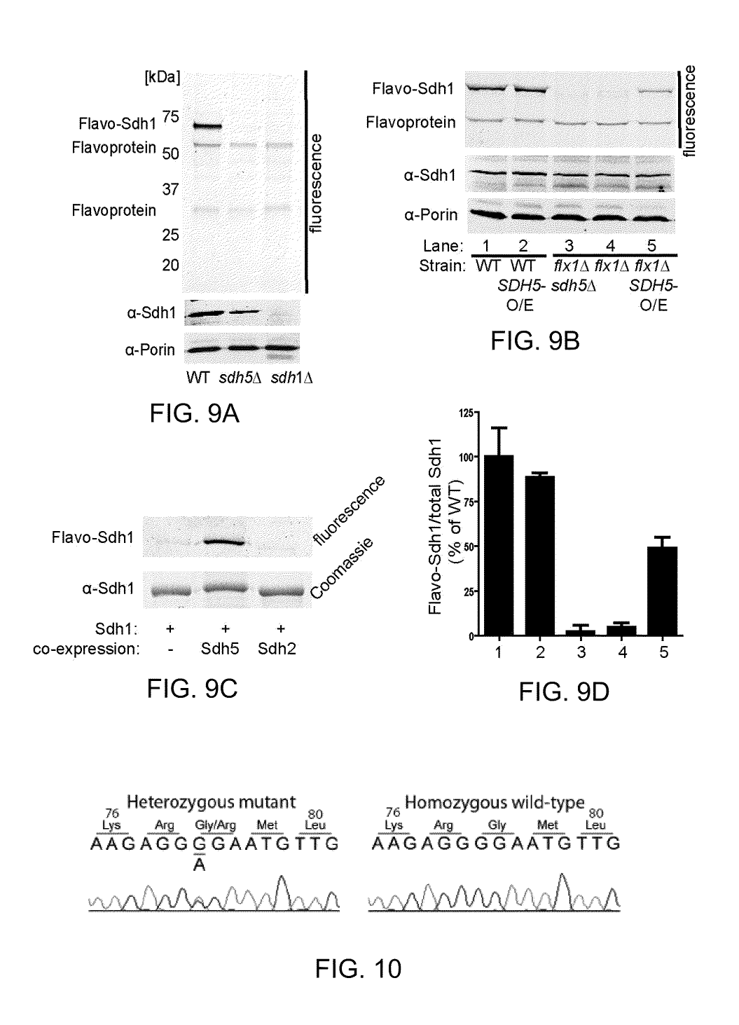

Sdh5 is necessary and sufficient for flavination of Sdh1 Multiple cofactors are required for the function of the SDH complex: FAD in Sdh1, an iron-sulfur cluster in Sdh2, and heme shared by Sdh3 and Sdh4. The cofactor attachment in Complex II was examined to see if this could be a reason for the impaired Complex II activity and stability in the sdh5Δ mutant. Due to the direct interaction between Sdh1 and Sdh5, the FAD attachment (flavination) of Sdh1 was examined. Unlike most cofactors, FAD is inserted covalently in Sdh1 and, therefore, can be detected fluorometrically after separation of mitochondrial proteins by SDS-PAGE. Deletion of SDH5 caused a complete loss of the Sdh1-FAD conjugate, just as was observed in sdh1Δ mutant mitochondria (FIG. 9A). Consistent with FIG. 8F, Sdh1 was still present in the absence of Sdh5, albeit at a decreased level compared to wild-type. This remaining Sdh1, however, exhibited no FAD fluorescence, demonstrating that Sdh5 is required for Sdh1 flavination. This Sdh5 requirement is specific to Sdh1, because the flavination of two other mitochondrial flavoproteins detected in the same gel was unaffected by either SDH5 or SDH1 deletion.

To address the sufficiency of Sdh5 for Sdh1 flavination, Sdh5 was overexpressed in wild-type cells using a high-copy 2μ plasmid, but an increase in Sdh1 flavination was not observed (FIG. 9B, lane 1 versus lane 2). This is likely due to already stoichiometric Sdh1 flavination in normal conditions, therefore, it can be reasoned that a state of reduced Sdh1 flavination might be required to observe an effect of Sdh5 overexpression. It has been reported that flavination of Sdh1 is compromised upon loss of the FAD transporter, Flxl, due to decreased total mitochondrial FAD. It was confirmed that the flx1Δ mutant exhibits severely decreased Sdh1 flavination (FIG. 9B, lane 4). When Sdh5 was overexpressed in flx1Δ mutant cells, however, Sdh1 flavination (normalized to Sdh1 protein level) was increased to about 50% of wild-type levels (FIG. 9B, lane 5). It should be noted that flavination of a distinct mitochondrial flavoprotein seen in the gel is not affected by FLX1 or SDH5 deletion or by SDH5 overexpression.

To further demonstrate the sufficiency of Sdh5 for Sdh1 flavination, Sdh1 was expressed in E. coli, either alone or in the presence of Sdh2 or Sdh5, and the flavination state of Sdh1 was examined. As shown in FIG. 9C, when Sdh1 was expressed alone, FAD incorporation was almost undetectable. When Sdh5 was co-expressed, however, flavination of Sdh1 was dramatically increased. This is not due simply to the overexpression of an Sdh1-interacting protein, as flavination did not increase when Sdh2 was co-expressed. These data demonstrate that Sdh5 is both necessary and sufficient for Sdh1 flavination.

Functional Conservation of Human SDH5 and Association with Paraganglioma

The amino acid sequence of yeast Sdh5 is 44% identical (from residue 33-158 of 163 total residues) to its human ortholog (SDH5), encoded by a previously uncharacterized gene Cllorf79. Cllorf79 will be referred to hereafter as hSDH5 to reflect its relationship with yeast SDH5. Based on the degree of sequence conservation, it can be hypothesized that hSDH5 will similarly be required for flavination of SDHA (the human ortholog of Sdh1) and thus for SDH activity.

Loss of SDH activity has been implicated in several neurological disorders and tumors, including paraganglioma (PGL). PGLs are neuroendocrine tumors derived from the extra-adrenal paraganglia of the autonomic nervous system. Four distinct familial PGL syndromes (PGL1-4) have been identified genetically, and PGL1, PGL3 and PGL4 have been associated with mutations in SDHD, SDHC and SDHB, respectively. The gene for PGL2 (OMIM accession# %601650) remains unidentified but was localized to a chromosomal region between the polymorphic markers D11S956 and PYGM, at chromosome 11q12.1 and 11q13.1, respectively. The hSDH5 gene is located almost exactly in the middle of this interval (NCBI Build 36.1 of the March 2006 human reference sequence) and is therefore a strong candidate for the PGL2 gene given its presumed role in SDH assembly and function.

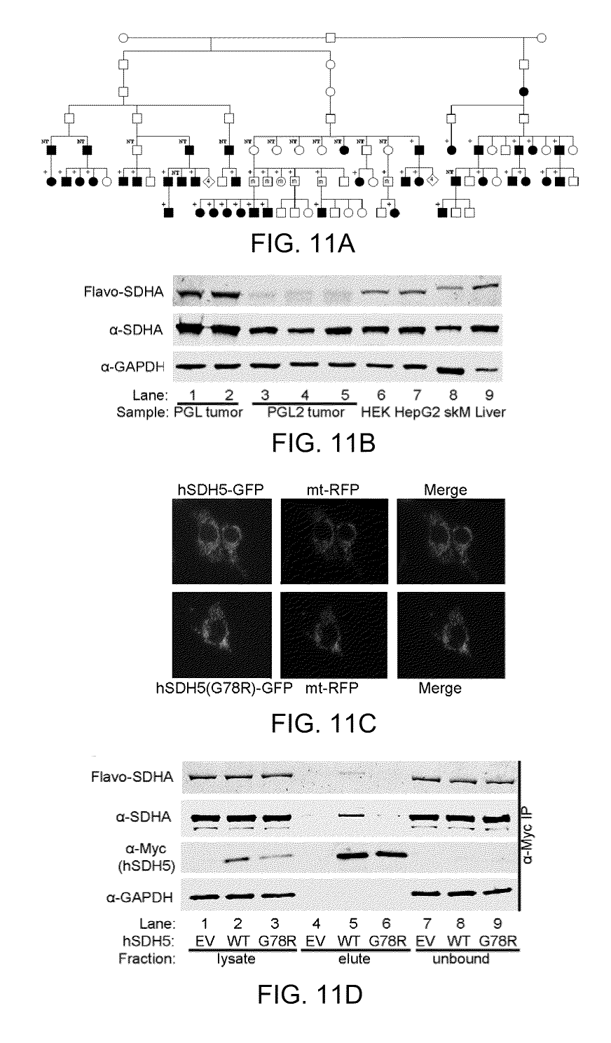

Mutation analysis was conducted in three affected PGL2 individuals from different branches of the previously described Dutch lineage by DNA sequencing of all four exons and exon-intron boundaries of hSDH5. A single nucleotide change was found in all three individuals, c.232G>A in exon 2 (FIG. 10; SEQ ID 13; SEQ ID 14), which causes a glycine to arginine substitution at residue 78 of the protein (FIG. 1). This substitution also introduces a BseRI restriction site in the mutated DNA sequence and BseRI digestion of the PCR fragment of exon 2 was performed for 400 control individuals. None of these individuals carried the c.232G>A mutation. Subsequently, the segregation of the mutation in the family was addressed by sequence analysis. The mutation was found to co-segregate with the disease haplotype for all 44 individuals that were known to have inherited this haplotype. Thirty-three individuals with the mutation have developed the disease, but not the seven individuals (median age 2009=74 yrs) that inherited the mutation from their mother (FIG. 11A). This suggests a mechanism similar to that described for SDHD (PGL1) in which SDHD mutations show very high penetrance upon paternal inheritance, while mutation carriers via the maternal line remain tumor-free throughout life. (See Baysal et al., (2000), Mutations in SDHD, a mitochondrial complex II gene, in hereditary paraganglioma, Science 287, 848-851; and van der Mey et al., (1989), Genomic imprinting in hereditary glomus tumours: evidence for new genetic theory. Lancet 2, 1291-1294, both of which are incorporated herein by reference.) A model explaining this mechanism has been proposed in which an imprinted and maternally expressed gene on chromosome 11p15.5 must be lost together with the normal maternal SDHD allele (the paternal allele is already inactivated by a germline mutation) prior to initiation of tumorigenesis (See Hensen et al., (2004), Somatic loss of maternal chromosome 11 causes parent-of-origin-dependent inheritance in SDHD-linked paraganglioma and phaeochromocytoma families, Oncogene 23, 4076-4083, incorporated herein by reference). This model can now be extended to the hSDH5 gene, apparently only the second tumor suppressor gene known to result in an “imprinted” phenotype. Only five individuals (median age 2009=42 yrs) that have the mutation on the paternal copy of the gene have not developed paragangliomas to our knowledge. As penetrance of the disease increases with age, these individuals may develop tumors in the future or tumors may already be present but undetected. These individuals are not marked in the pedigree for privacy reasons. None of the 44 members of the family that did not inherit the disease haplotype were found to carry the mutation nor did any of the 9 spouses tested.

The mutated G78 is part of an arginine-glycine dipeptide that is conserved in all eukaryotic and prokaryotic species for which sequence information is available and is in the most highly conserved region of the protein (FIG. 1). Further, a glycine to arginine transition is a dramatic alteration in amino acid side chain chemistry. Next it was examined whether the G78R mutation would abolish hSDH5 function and as a result cause severely decreased SDHA flavination in PGL2 tumor samples. Because normal carotid body, the site of origin for these tumors, was unavailable as a control, we examined flavination in tumors from sporadic PGL patients (non-PGL2), cultured human cells, and normal mouse tissues. SDHA flavination was decreased (˜95%) in tumors from three separate PGL2 patients carrying the hSDH5 G78R mutation compared to tumors from two sporadic PGL patients, cultured human cells and mouse tissues (FIG. 11B).

Constructs expressing wild-type and G78R hSDH5 were generated as C-terminal GFP and Myc fusions. In human HEK293 cells, both the wild-type and mutant GFP-fusion proteins localized exclusively to mitochondria, showing that neither expression nor mitochondrial localization is compromised by this mutation (FIG. 11C). The expression and SDHA interaction of the Myc-tagged wild-type and G78R hSDH5 proteins were also examined. As shown in FIG. 11D, immunoprecipitation of wild-type hSDH5 causes SDHA to be precipitated, showing that the Sdh1-Sdh5 interaction observed in yeast is conserved in mammalian cells. The protein level of the G78R mutant of hSDH5 is lower than that of the wild-type protein, suggesting the mutant protein is at least partially unstable. The remaining G78R hSDH5 appears to have the additional defect of impaired SDHA interaction (FIG. 11D). It can be conclude, therefore, that the G78R mutation is likely to cause at least two defects; protein instability and impaired SDHA interaction. It is also of note that the proportion of SDHA that is associated with hSDH5 (FIG. 11D, lane 5) has the same proportional flavination as bulk SDHA or SDH5-free SDHA (FIG. 11D, lanes 2 and 8). Therefore, SDH5 appears to interact with both flavinated and un-flavinated SDHA.

The hSDH5 G78R is a loss of Function Mutation

To further understand the nature of the G78R mutation in hSDH5, the ability of wild-type and G78R mutant hSDH5, both expressed from the yeast SDH5 promoter, to complement the glycerol growth defect of the sdh5Δ mutant strain was assessed. As shown in FIG. 12A, wild-type hSDH5 enabled growth similar to yeast Sdh5, but the G78R mutant failed to do so and was indistinguishable from the empty vector. Overexpression of any of the individual SDH subunits or the proposed SDH complex chaperone TCM62 failed to complement the sdh5Δ mutant phenotype (FIG. 13). Regarding FIG. 13, serial 5-fold dilutions of saturated SD-Ura liquid cultures of sdh5Δ mutant strain transformed with 2μ high-copy plasmid expressing Sdh1-4 subunits or Tcm62 (a SDH complex chaperone) were spotted on S-URA solid medium containing 2% glucose or 3% glycerol as carbon source and grown at 30° C. for 2 days (glucose) or 3 days (glycerol). Further, an extensive high-copy suppressor screen of the sdh5Δ glycerol growth defect using a yeast genomic library recovered many independent isolates of SDH5, but failed to identify any other suppressing genes. Therefore, the specific rescue of the sdh5Δ glycerol growth defect by heterologous expression of hSDH5, from the native yeast SDH5 promoter, is strong evidence of the conservation of Sdh5 function from yeast to man.

The Sdh1 flavination in the same five strains used for the above glycerol growth test was then directly measured. As shown in FIG. 12B, Sdh1 flavination in sdh5Δ mitochondria is restored by a plasmid-borne yeast SDH5 gene. Expression of hSDH5 increases the flavination of yeast Sdh1 to about 75% of the wild-type level after normalization to total Sdh1 protein level, but the G78R mutant has no effect on Sdh1 flavination. The loss of genetic function of the G78R mutant in this assay is likely due to a near complete destabilization of the protein. Wild-type hSDH5 accumulated in mitochondria to a level near that of yeast Sdh5, the G78R mutant was nearly undetectable (FIG. 12B). Combined with the decreased protein level observed in FIG. 11D, these data indicate that the G78R mutation causes destabilization and degradation of hSDH5 in addition to other possible defects, including weakened SDHA interaction.

Further Discussion

The molecular mechanism of covalent FAD insertion into proteins has previously proven difficult to elucidate. Other covalent cofactors are inserted into proteins by specialized enzymes, such as cytochrome c heme lyase for heme attachment to apocytochrome c. Until now, it appears that no similar enzyme has been identified for covalent FAD attachment. Covalent FAD attachment to Sdh1 appears to be dependent on additional protein factors. Using in vitro translated Sdh1 precursor and differentially treated mitochondria, Sdh1 flavination was shown to require matrix import, ATP and at least one additional protein (See Robinson et a., (1996), A requirement for matrix processing peptidase but not for mitochondrial chaperonin in the covalent attachment of FAD to the yeast succinate dehydrogenase flavoprotein, J Biol Chem 271, 4061-4067, incorporated herein by reference.) It is possible that Sdh5 is the necessary matrix protein suggested by these earlier experiments.

The question remains, however, whether Sdh5 actually participates in the chemistry of FAD attachment (enzymatic function) or simply maintains Sdh1 in a conformation that is susceptible to autocatalytic FAD attachment (chaperone function). Four observations would suggest that Sdh5 does not act simply as a general chaperone. First, in addition to the myriad of native chaperones in both the mitochondrial matrix of S. cerevisiae and in E. coli, co-expression of Sdh2, a clear Sdh1 binding partner, is unable to support flavination of Sdh1 in either yeast or bacteria (FIG. 9A, 9C, and FIG. 13). Second, as shown in FIG. 11D, hSDH5 interacts quite robustly with SDHA. If hSDH5 were acting simply as a chaperone to promote FAD incorporation, it is likely that the hSDH5-associated population of SDHA would not be flavinated. The present studies have found, however, that the hSDH5-associated SDHA has the same normalized FAD signal as the hSDH5-unbound SDHA. Third, an extensive high-copy suppressor screen was conducted for rescue of the sdh5Δ glycerol growth defect. Many independent SDH5-containing plasmids were recovered, but no additional genes were found that enabled bypass of SDH5. This is suggestive of a fairly specific role for Sdh5 that cannot be compensated by increased general chaperone activity (or by other means). Fourth, the sequence of Sdh5 may be suggestive of a direct relationship with FAD. The most highly conserved region of Sdh5 (surrounding the G78 position) is highly basic in all species (FIG. 1). It is possible that this region participates in an interaction with the negatively charged pyrophosphate moiety of FAD during the attachment reaction. The high conservation of this basic region extends throughout all branches of eukaryotes and to some bacterial species, including Rickettsia. Rickettsia is thought to be related to the bacterium that entered the endosymbiotic relationship with a eukaryotic cell and became the ancestral mitochondrion. The divergent N-terminus may be involved in mitochondrial targeting, where sequence identity is less critical for function.

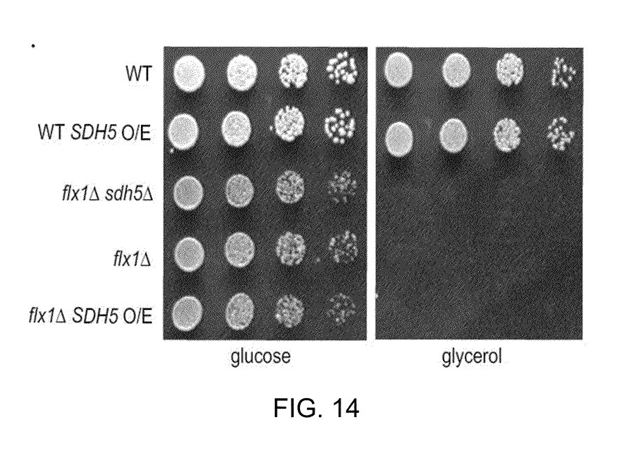

Whatever the exact role of Sdh5 in Sdh1 flavination, three observations suggest that it plays that role very specifically. First, as shown in FIG. 6B, in the absence of Sdh1, the Sdh5 protein fails to accumulate. The fact that loss of Sdh1 leads to a complete loss of Sdh5, presumably through protein destabilization and degradation, suggests that Sdh1 is an obligate binding partner of Sdh5. This intimate relationship implies a high degree of specificity for Sdh5 function. Second, Sdh5 overexpression in the flx1Δ mutant strain lacking the mitochondrial FAD transporter enhanced Sdh1 flavination, but failed to rescue the glycerol growth defect of the flx1Δ mutant strain (FIG. 14), which is presumably caused by deficiency of FAD occupancy in other flavoproteins. Regarding FIG. 14, serial 5-fold dilutions of saturated SD-Ura liquid culture of each indicated strain (the same five strains described in FIG. 9B) were spotted on S-URA solid medium containing 2% glucose or 3% glycerol as carbon source and grown at 30° C. for 2 days (glucose) or 3 days (glycerol). Third, covalent FAD attachment to the two other mitochondrial proteins visualized in the fluorescent gel assay was completely normal in the sdh5Δ mutant, and it was not affected by Sdh5 overexpression (FIGS. 9A and 9B). Although the identities of these two mitochondrial flavoproteins were not experimentally determined, lipoamide dehydrogenase (Lpd1/Yfl018c) is likely to be the one that migrates slightly faster than Sdh1 at 50 kD. The Lpd1 covalent flavoprotein localizes to the mitochondrial matrix, the same localization as Sdh5. The fact that FAD covalent attachment to Lpd1 is completely unaffected by Sdh5 deletion or overexpression is, therefore, a strong indicator of the specificity of Sdh5 function. It is likely that FAD attachment factors are important for other flavoproteins, although they do not bear obvious primary sequence identity to Sdh5.

A surprising observation that has been consistently corroborated is that paraganglioma is associated with mutations in SDHB, SDHC and SDHD, but not with SDHA. SDHA mutations, but not SDHB, SDHC and SDHD mutations, are associated with Leigh syndrome. If these proteins function only when together as a complex, why should their mutation lead to different outcomes? One intriguing possibility is suggested by three observations shown in FIGS. 6B and 8F. First, Sdh1 and Sdh2 form a dimer and are mutually required for normal protein stability. Sdh2 is completely degraded in an sdh1Δ mutant strain. Sdh1, on the other hand, exhibits reduced protein accumulation in the sdh2Δ mutant, but is not completely degraded (FIG. 6B). Second, in the absence of Sdh5, every subunit of the SDH complex has decreased protein abundance (presumably degradation due to destabilization of the complex), but the least affected subunit is Sdh1, the one that is directly bound by Sdh5 (FIG. 8F). Finally, the excess Sdh1, which accumulates in the sdh5Δ mutant, is found in soluble form in the mitochondrial matrix, while all of the detectable Sdh2, Sdh3 and Sdh4 remains membrane-associated (FIG. 8F). These data clearly demonstrate that Sdh1 has the unique ability to exist stably in the absence of the other SDH subunits and SDH complex assembly, and this is not dependent on the specific Sdh1-interacting protein, Sdh5. We raise the very speculative possibility that a minor population of Sdh1/SDHA acts in an additional capacity that is independent of the other complex subunits and is also independent of FAD incorporation (as SDH5 mutations cause PGL). It is the loss of this secondary function that leads to severe early-onset neurological disorders such as Leigh syndrome before the presentation of PGL, which typically presents at an older age.

There are immediate clinical implications of this work. It has been estimated that roughly 70% of familial cases of head and neck PGL are due to germline mutations in SDHB, SDHC or SDHD (See Baysal et al., (2002), Prevalence of SDHB, SDHC, and SDHD germline mutations in clinic patients with head and neck paragangliomas, J Med Genet 39, 178-183, incorporated herein by reference). It is likely that germline mutations in hSDH5 are causative in many of the remaining 30% of cases. Similarly, recent studies found that 10% of sporadic PGL was associated with mutations in SDH complex subunits (6% SDHB, 4% SDHD) (See Schiavi et al., (2005), Predictors and prevalence of paraganglioma syndrome associated with mutations of the SDHC gene, Jama 294, 2057-2063; and Timmers et al., (2009), Clinical aspects of SDHx-related pheochromocytoma and paraganglioma, Endocr Relat Cancer.; both of which are incorporated herein by reference). It is likely that hSDH5 mutations will also be found in a subset of the remaining 90% of sporadic PGL. Recently, the Carney-Stratakis syndrome has been identified, implicating mutations in SDH complex genes (SDHB, SDHC and SDHD) in gastrointestinal stromal tumors in addition to PGL (See McWhinney et al., (2007), Familial gastrointestinal stromal tumors and germ-line mutations, N Engl J Med 357, 1054-1056; and Pasini et al., (2008), Clinical and molecular genetics of patients with the Carney-Stratakis syndrome and germline mutations of the genes coding for the succinate dehydrogenase subunits SDHB, SDHC, and SDHD, Eur J Hum Genet 16, 79-88; both of which are incorporated herein by reference). Mutations in hSDH5 might also be causal in this new syndrome. Defects in SDH5 might also be involved in the development of pheochromocytomas, which are often associated with mutations in SDHB, SDHC and SDHD (Timmers et al., 2009). The identification of a new gene, the mutation of which is likely causal in a subset of patients, enables more comprehensive genetic testing. It has been suggested that the clinical management of PGL, even in the absence of evidence for familiality, should involve genetic testing (Cascon et. Al., (2009), Rationalization of Genetic Testing in Patients with Apparently Sporadic Pheochromocytoma/Paraganglioma, Horm Metab Res, incorporated herein by reference) and this genetic testing regime should now be modified to include hSDH5. This enables early identification and diagnosis of affected relatives, leading to improved morbidity and mortality outcomes. Other forms of cancer and a variety of other diseases may also be due to hSDH5 mutations (See Ricketts et al., (2008), Germline SDHB mutations and familial renal cell carcinoma, J Natl Cancer Inst 100, 1260-1262; and Birch-Machin et al., (2000), Late-onset optic atrophy, ataxia, and myopathy associated with a mutation of a complex II gene, Ann Neurol 48, 330-335; both of which are incorporated herein by reference).

### EXAMPLES

### Example 1

### Yeast Strains

Saccharomyces cerevisiae haploid strain JRY472 (W303a MATa his3 leu2 met15 trpl ura3) was used as the parental and wild-type strain. Deletion mutant strains were generated by KanMX4 disruption using homologous recombination in JRY472 except that sdh5Δ mutant haploid was derived from tetrad dissection of the heterozygous diploid. For all the double mutant strains, the sdh5Δ::KanMX4 mutant strain was first converted to NatMX4 by homologous recombination and then used for KanMX4 deletion of the second gene. The Sdh5-TAP strain was created by integration of the TAP tag in frame downstream of SDH5 in the genome of JRY472. All engineered strains were verified by PCR. Complete strain information is provided in Table 1. Yeast cells were transformed by the standard lithium acetate method and cultured in either rich (YP) medium or synthetic complete (SC) medium lacking the appropriate amino acids for plasmids selection, containing either 2% glucose, 2% raffinose or 3% glycerol carbon source as indicated.

TABLE 1
Yeast Strains

| Strain | Genotype |
|---|---|
| JRY472 (W303) | MATa his3 leu2 lys2 met15 trp1 ura3 |
| JRY597 | MATa his3 leu2 lys2 met15 trp1 ura3 |
| | sdh5::SDH5-TAP-KanMX4 |
| JRY609 | MATa his3 leu2 lys2 met15 trp1 ura3 sdh5::KanMX4 |
| JRY989 | MATa his3 leu2 lys2 met15 trp1 ura3 sdh5::NatMX4 |
| JRY991 | MATa his3 leu2 lys2 met15 trp1 ura3 sdh1::KanMX4 |
| JRY992 | MATa his3 leu2 lys2 met15 trp1 ura3 sdh2::KanMX4 |
| JRY993 | MATa his3 leu2 lys2 met15 trp1 ura3 sdh3::KanMX4 |
| JRY994 | MATa his3 leu2 lys2 met15 trp1 ura3 sdh4::KtmMX4 |
| JRY995 | MATa his3 leu2 lys2 met15 trp1 ura3 tcm62::KanMX4 |
| JRY996 | MATa his3 leu2 lys2 met15 trp1 ura3 sdh1::KanMX4 |
| | sdh5::NatMX4 |
| JRY997 | MATa his3 leu2 lys2 met15 trp1 ura3 sdh2::KanMX4 |
| | sdh5::NatMX4 |
| JRY998 | MATa his3 leu2 lys2 met15 trp1 ura3 sdh3::KanMX4 |
| | sdh5::NatMX4 |
| JRY999 | MATa his3 leu2 lys2 met15 trp1 ura3 sdh4::KanMX4 |
| | sdh5::NatMX4 |
| JRY1070 | MATa his3 leu2 lys2 met15 trp1 ura3 flx1::KanMX4 |
| | sdh5::NatMX4 |

### Example 2

### Cell Lines and Tumor Samples

HEK293 cells and HepG2 cells were obtained from ATCC and maintained in DMEM supplemented with 10% FBS and antibiotics. HEK293 cells were transfected in suspension using Fugene 6 reagent (Roche) following the manufacturer's instructions. Frozen PGL tumor samples were obtained from affected patients with informed consent.

### Example 3

### PGL2 Family

The family in which the PGL2 locus was identified (Mariman et al., (1995), Fine mapping of a putatively imprinted gene for familial non-chromaffin paragangliomas to chromosome 11q13.1: evidence for genetic heterogeneity, Hum Genet 95, 56-62; and Mariman et al., 1993; both of which are incorporated herein by reference) has been previously described (van Baars et al., (1982), Genetic aspects of nonchromaffin paraganglioma, Hum Genet 60, 305-309, incorporated herein by reference). A significant number of additional family members have been clinically evaluated in the Department of Otorhinolaryngology of the Radboud University Hospital and segregation of the disease haplotype performed as described by Mariman (Mariman et al., 1995). These studies were approved by local ethics committees for the Radboud University Nijmegen Medical Centre and the Leiden University Medical Centre.

### Example 4

### Plasmid Construction

All yeast expression plasmids used in this study were constructed by ligation of a PCR product containing the promoter and coding region of the relevant gene into a pRS416-based vector containing a C-terminal GFP, His-HA₃ or His-Myc₂ tag and a UGP1 terminator, or into pRS426 vector for overexpression as indicated. The integrity of the insert and functionality of the fusion protein were verified by DNA sequencing and rescue of the phenotype of the corresponding deletion strain, respectively. For bacterial expression of His-tagged yeast Sdh1, Sdh1 coding sequence was subcloned into pRSFDuet vector (Novagen) containing an N-terminal His tag. Sdh2-His-Myc and Sdh5-His-HA coding sequences were subcloned from yeast expression constructs into pCDFDuet and pETDuet, respectively. These pDuet vectors contain compatible replicons and drug resistance for co-expression. For expression of human SDH5 in yeast under control of the yeast SDH5 promoter, the yeast SDH5 promoter and human SDH5 coding sequence cloned from HepG2 cDNA were sequentially ligated into the Myc-tag containing pRS416 vector as described above. hSDH5 mammalian expression plasmids were generated by ligation of hSDH5 cDNA into a pcDNA3-based vector containing a C-terminal GFP or Myc tag. hSDH5 G78R mutants were generated from wild-type constructs using a QuickChange site-directed mutagenesis kit (Stratagene). pYX142-TPI-mtRFP yeast expression plasmid and pDsRed2-Mito mammalian expression plasmid used to visualize mitochondria were gifts of Dr. Janet Shaw and Dr. Kevin Flanigan at the University of Utah, respectively.

### Example 5

### Mitochondria Purification and Assays

Intact mitochondria were isolated from yeast by differential centrifugation after cell wall removal by Zymolyase (US Biological) treatment and cell lysis by douncing as described previously (Diekert et al., (2001), Isolation and subfractionation of mitochondria from the yeast Saccharomyces cerevisiae, Methods Cell Biol 65, 37-51, incorporated herein by reference). Mitochondrial protein concentration was determined by Advanced Protein Assay Reagent (Cytoskeleton, Inc). Proteinase K protection assay for sub-mitochondrial localization study was performed as previously described (Diekert et al., 2001). Mitochondrial membranes were isolated by sonication of mitochondria in 20mM Hepes buffer pH 7.4, followed by ultracentrifugation at 100,000 g for 30 min for the membrane association assay. The enzymatic activities of succinate dehydrogenase and malate dehydrogenase were measured spectrophotometrically with isolated mitochondria as described (Chen et. Al., (2002), Inhibition of Fe—S cluster biosynthesis decreases mitochondrial iron export: evidence that Yfh1p affects F—S cluster synthesis, Proc Natl Acad Sci U S A 99, 12321-12326, incorporated herein by reference) and normalized to total mitochondrial protein.

### Example 6

### Oxygen Consumption Assay

Yeast strains were grown to mid-log phase in SRaff (2% raffinose) liquid media and 2 OD of each culture was resuspended in 3% glycerol solution after washing with sterile water. The rate of oxygen consumption was measured using a 5300A Biological Oxygen Monitor (Yellow Springs Instrument Co.). The linear region of data was used for calculation.

### Example 7

### Tandem Affinity Purification

The nickel chromatography and anti-HA affinity chromatography two-step purification was performed as described (Honey et al., 2001), except that purified mitochondria and commercial anti-HA conjugated agarose beads (Sigma) were used for purification and elution was achieved by 200 ng/ml HA peptide. The final eluates were precipitated in 10% TCA and 200ug/m1 sodium deoxycholate, resolved using 15% SDS-PAGE and visualized by SilverSNAP Stain (Pierce) or immunoblot with indicated antibodies. Validated samples were then subjected to LC-MS-MS for protein identification.

### Example 8

### Co-Immunoprecipitation

At 30 h post-transfection, HEK293 cells grown on 10 cm plates were harvested by trypsinization, washed with PBS and frozen at −80° C. Cells were then resuspended in 500 ul binding buffer (10 mM Tris-HCl pH7.4, 150 mM NaCl, 0.1% NP-40, 0.5 mM EDTA and 10% glycerol), passed through an insulin syringe 10 times to shear DNA, and centrifuged at 14,000 rpm for 10 min at 4° C. to remove cell debris. Cleared lysates were incubated with equilibrated anti-Myc conjugated agarose beads (Sigma) at 4° C. for 2 h. After extensive washing, bound proteins were eluted in 1×SDS sample buffer for immunoblot.

### Example 9

### Immunoblot

Yeast proteins were extracted by a NaOH-based method as described (Kushnirov, V.V. (2000), Rapid and reliable protein extraction from yeast, Yeast 16, 857-860, incorporated herein by reference) or by directly dissolving purified mitochondria in 1×SDS sample buffer. Tissue lysates were prepared by homogenizing approximately 30 mg frozen human PGL tumor or mouse tissue in cell lysis buffer (Cell Signaling Technology) with a Tissue-Tearer rotor after pulverization by Bessman tissue pulverizer. HEK293 and

HepG2 cell lysates were prepared by sonication on ice of harvested cells in above cell lysis buffer. After centrifugation at 14,000 rpm for 20 min at 4° C., cleared lysates were collected to measure protein concentration. Protein samples were then separated by SDS-PAGE and transferred to nitrocellulose for blocking and incubation with primary antibody and secondary antibody conjugated with infared dyes for detection by the Odyssey system (LI-COR). Quantitation of protein bands was performed using the built-in Odyssey software. The sources of primary antibodies are indicated: anti-HA (Roche); anti-Myc (Covance); anti-Porin and anti-PGK (Invitrogen); anti-SDHA (Mitosciences); anti-GAPDH (Chemicon); anti-Mge1 and anti-Fzo1 (Janet Shaw, University of Utah); anti-Aco1 (Jerry Kaplan, University of Utah); anti-Sdh1 and anti-Sdh2 (Bernard Lemire); anti-Tim10 (Carolyn Outten).

### Example 10

### Sdh1/SDHA Flavination Assay

The flavination assay is adapted from a UV-transilluminator method described previously (Bafunno et al., (2004), Riboflavin uptake and FAD synthesis in Saccharomyces cerevisiae mitochondria: involvement of the Flxlp carrier in FAD export, J Biol Chem 279, 95-102, incorporated herein by reference). After SDS-PAGE separation of mitochondrial proteins or whole cell extracts, the protein gel was incubated for 20 min in 10% acetic acid to adjust pH. 526 nm fluorescence emission by covalently bound FAD upon excitation at 488 nm was measured by the Typhoon imager (GE Healthcare). Quantitation of fluorescence was done by densitometry using AlphaEaseFC™ software.

### Example 11

### Blue Native-PAGE and Applications

BN-PAGE was performed essentially as described (Wittig et al., (2006), Blue native PAGE, Nat Protoc 1, 418-428; Pierrel et al., (2007), Coal links the Mss51 post-translational function to Coxl cofactor insertion in cytochrome c oxidase assembly, Embo J 26, 4335-4346; both of which are incorporated herein by reference), with slight modification. In-gel activity assay of respiratory complexes after BN-PAGE was performed as previously described (Zerbetto et al., (1997), Quantification of muscle mitochondrial oxidative phosphorylation enzymes via histochemical staining of blue native polyacrylamide gels, Electrophoresis 18, 2059-2064; Jung et al., (2000), Measuring the quantity and activity of mitochondrial electron transport chain complexes in tissues of central nervous system using blue native polyacrylamide gel electrophoresis, Anal Biochem 286, 214-223; both of which are incorporated herein by reference). Coomassie stain and immunoblot were performed according to standard protocols for SDS-PAGE, except that 0.1% SDS is supplemented in the transfer buffer to facilitate protein transfer.

### Example 12

### Fluorescence Microscopy

The sdh5Δ mutant strain transformed with both pSdh5-GFP and pMito-RFP plasmids was grown to mid-log phase medium containing 2% glucose or 2% raffinose and imaged using a Zeiss Axioplan 2 Imaging microscope as described (Kondo-Okamoto et. Al., (2003), Mmmlp spans both the outer and inner mitochondrial membranes and contains distinct domains for targeting and foci formation, J Biol Chem 278, 48997-49005. Epub 42003 Sep 48912, incorporated herein by reference). Human HEK293 cells were transfected with pDsRed2-Mito (Mito-RFP) and pcDNA3-hSDH5-GFP or pcDNA3-hSDH5(G78R)-GFP in 35mm plates. At 24 h post transfection, cells were photographed using the Olympus IX81 microscope at the University of Utah Fluorescence Microscopy Core Facility.

### Example 13

### Protein Expression and Purification

BL21(DE3) CodonPlus-RIL (Stratagene) E. coli strain was transformed with pRSFDuet-His-Sdh1 alone or together with pCDFDuet-Sdh2-Myc or pETDuet-Sdh5-HA. 0.2 mM final concentration of IPTG was added to 1 liter culture of each strain at OD=0.8 to induce protein expression for 6 h at 37° C. The cells were harvested and His-tagged proteins were purified using Ni-NTA agarose beads (Qiagen) according to the manufacturer's protocol.

### Example 14

### Mutation Analysis

Primers for amplification of exons and exon-intron boundaries of the Cllorf79 gene (Ensembl gene number ENSG00000167985; http://www.ensembl.org/index.html) were designed using ExonPrimer (http://ihg2.helmholtz-muenchen.de/ihg/ExonPrimer.html) and the reference sequence NT_{—}033903. Primer sequences are provided in Table 2. (SEQ ID 15-24). For amplification, 50 ng of genomic DNA was used as the starting material and primer annealing was performed at 58° C. The MgCl₂ concentration in the reaction mix was 2 mM and for amplification of exon 1, 10% DMSO was added.

PCR fragments were purified using NucleoFast 96 PCR plates (Clontech). Sequence analysis was performed with the ABI PRISM Big Dye Terminator Cycle Sequencing V2.0 Ready Reaction kit and the ABI PRISM 3730 DNA analyzer (Applied Biosystems). As a reference sequence NM017841 was used.

For the presence of the mutation in control individuals, exon 2 was amplified as described for sequencing. The fragments were purified with NucleoFast 96 PCR plates (Clontech) and digested with BseRl (New England Biolabs) according to manufacturer's protocols. Restriction fragments were analyzed on 2% agarose gels.

TABLE 2
Primer Sequences for Amplification of hSDH5 exons

| Exon | forward primer | reverse primer |
|---|---|---|
| Exon 1 | 5′-ACCTTCCGGCTCAGCTC-3′ | 5′-TATCGGGCAGACGAACTC-3′ |
| Exon 2 | 5′-GTTGACCTTCCCAGGCTC-3′ | 5′-GAGGTTCAGCTGCTTTTCTG-3′ |
| Exon 3 | 5′-GGTTCAGAGAGACTCCCAGG-3′ | 5′-GCAACGAGAGTGAAACTCAG-3′ |
| Exon 4-1 | 5′-CCCTGGTATAGGCTAACATCG-3′ | 5′-TGAGTACACTTGGGCTGAGG-3′ |
| Exon 4-2 | 5′-AGCTCTGAGCCTCAAAAGTG-3′ | 5′-GAAGACTGTAGGAATGAGGGG-3′ |

### Example 15

### Sdh5 is a Soluble Mitochondrial Matrix Protein

The sdh5Δ strain containing both a plasmid expressing Sdh5-GFP under the native SDH5 promoter and a plasmid expressing mito-RFP (a fusion of the N. crassa F0-ATPase Su9 presequence to RFP) was grown in SD-Ura-Leu and harvested at mid-log phase for fluorescence microscopy. A representative population of cells is shown in FIG. 3A. Whole cell extract (WCE) from sdh5Δ strain transformed with a plasmid expressing Sdh5-HA under the native SDH5 promoter was centrifuged at 12,000 g for 10 min to yield the mitochondrial pellet (Mito) and post-mitochondria supernatant (PMS). These fractions were analyzed by immunoblotting with the indicated antibodies (Porin: mitochondrial marker; PGK: cytoplasmic marker) (See FIG. 3B). Mitochondria, mitoplasts generated by hypotonic swelling, and 1% Triton X-100 solubilized mitochondria purified from the strain shown in FIG. 3B, were treated with (+) or without (−) proteinase K and analyzed by immunoblotting along with an untreated mitochondria control (UT). The sub-mitochondrial localization of Mge1, Tim10 and Fzo1 are matrix, intermembrane space and outer membrane, respectively. (See FIG. 3C). Purified mitochondria from the strain used in FIG. 3B were sonicated in 20 mM Hepes buffer pH 7.4 followed by ultracentrifugation at 100,000 g for 30 min. The soluble and membrane fractions were assayed by immunoblotting with the indicated antibodies. Aconitase 1 (Aco1) is a soluble matrix protein and succinate dehydrogenase subunit 1 (Sdh1) is a membrane-associated matrix protein. (See FIG. 3D).

### Example 16

### The sdh5Δ Mutant is Respiratory Deficient but has Intact Mitochondrial DNA

Wild-type and sdh5Δ mutant strains were transformed with empty vector (EV) or a plasmid expressing Sdh5-HA under control of the native SDH5 promoter. Serial 5-fold dilutions of saturated SD-Ura liquid culture of each strain were spotted on S-URA solid medium with either 2% glucose or 3% glycerol as the carbon source and grown at 30° C. for 2 days (glucose) or 3 days (glycerol). (See FIG. 2A). Wild-type, sdh5Δ and sdh1Δ strains were grown to mid-log phase in SRaff (2% raffinose) liquid media and the rate of oxygen consumption was measured in a 3% glycerol solution. Data represent the average±standard deviation of 3 replicates per strain. Raffinose allows growth of respiratory deficient strains, but stimulates respiration and mitochondrial biogenesis. Qualitatively similar results were obtained with strains grown in glucose. (See FIG. 2B). The sdh5Δ mutant haploid was mated with a mtDNA-deficient (rho⁰) haploid of the opposite mating type. Two independent resultant diploids and the parental haploids were streaked on S-URA plate with either 2% glucose or 3% glycerol as the carbon source and grown at 30° C. for 2 days (glucose) or 3 days (glycerol). (See FIG. 2C).

### Example 17

### Sdh5 Physically Interacts with Sdh1

Mitochondria were purified from 6 liter cultures of the wild-type strain or the sdh5Δ strain containing a plasmid expressing C-terminally His and HA-tagged Sdh5 under the native SDH5 promoter. Lysate from purified mitochondria was subjected to nickel chromatography and anti-HA affinity chromatography, finally eluting with 200 ng/ml HA peptide. As is shown in FIG. 6A, the final elutions were TCA precipitated, resolved using SDS-PAGE and visualized by silver staining (top panel) or immunoblot with α-Sdh1 and α-HA antibodies (lower panels). FIG. 6B shows an immunoblot of purified mitochondria from pSdh5-HA plasmid-containing sdh5Δ strain (WT), or the sdh1Δ sdh5Δ (sdh1Δ) or sdh2A sdh5Δ (sdh2A) double mutant strains. The mitochondrial protein Porin serves as a loading control.

### Example 18

### Sdh5 is Required for Succinate Dehydrogenase Complex Activity and Stability

Purified mitochondria from wild-type and sdh5Δ strains grown in YPA-Raff media were assayed for succinate dehydrogenase and malate dehydrogenase activity. Activity measurements were normalized to the total protein amount and expressed as a percentage relative to the wild-type strain. Data represent the average±standard deviation of 3 replicates per strain. (See FIG. 8A). FIG. 8B shows in-gel catalytic activity assay for respiratory complex II, IV and V after BN-PAGE separation of mitochondrial membrane fractions from wild-type and sdh5Δ mutant strains. V₂ denotes a complex V dimer. FIG. 8C shows BN-PAGE separation of mitochondrial membrane fractions from wild-type and sdh5Δ mutant strains followed by Coomassie blue staining. FIG. 8D shows an immunoblot of BN-PAGE separated respiratory complex II (or succinate dehydrogenase) using α-Myc antibody with wild-type and sdh5Δ mutant mitochondria wherein Sdh3 is Myc-tagged. FIG. 8E shows and immunoblot of BN-PAGE separated Sdh5 complex using PAP antibody with WT and Sdh5-TAP mitochondria, both without and with 1% SDS addition before electrophoresis. 440 kD Porin complex is both a loading and quality control. FIG. 8F shows purified mitochondria from wild-type (lanes 1, 3 and 4) and sdh5Δ mutant (lanes 2, 5 and 6) strains wherein Sdh3 or Sdh4 is Myc-tagged were separated into soluble (sol, lanes 3 and 5) and membrane (mem, lanes 4 and 6) fractions as described for FIG. 3D. These fractions together with the equivalent amount of unfractionated mitochondria (total, lanes 1 and 2) from both strains were immunoblotted with the indicated antibodies. Aconitase 1 (Aco1) is a soluble matrix protein serving as a loading control. The indicated percentage is the amount of each SDH subunit remaining in sdh5Δ mitochondria relative to that in wild-type mitochondria. The two percentages in the parentheses are percentage of soluble Sdh1 or Sdh2 in the wild-type (left) and sdh5Δ (right) mitochondria.

### Example 19

### Sdh5 is Necessary and Sufficient for Flavination of Sdh1

FIG. 9A shows wild-type, sdh5Δ and sdh1Δ mitochondria resolved by SDS-PAGE and a gel scanned for fluorescence (488 nm excitation, 526 nm emission) to detect covalently linked FAD (top panel). Immunoblotting with α-Sdh1 and α-Porin (loading control) antibodies was performed on the same samples (lower panels). FIG. 9B shows a fluorescence gel image (top panel) and immunoblotting as in FIG. 9A with whole cell extract from wild-type (lanes 1-2) and flx1Δ sdh5Δ mutant strains (lanes 3-5) containing CEN (˜1 copy/cell) or 2μ (˜10 copies/cell) empty vector (EV) or plasmid expressing Sdh5 under control of the SDH5 promoter. For each strain, FAD fluorescence was normalized to Sdh1 protein level and expressed as a percentage relative to wild-type (bottom panel). Data represent the average±standard deviation of 3 replicates per strain. FIG. 9C shows His-tagged yeast Sdh1 was expressed either alone or with Sdh5 or Sdh2 in E. coli and purified by nickel chromatography. The purified Sdh1 was then analyzed for FAD fluorescence as in (A) and by Coomassie blue staining.

### Example 20

### Human SDH5 is Mutated in a Dutch Lineage with Paraganglioma

FIG. 11A shows the segregation of the c.232G>A transition in the previously described PGL2 family. The heterozygous mutation segregates with the disease. +, mutation heterozygously present; m, mutation inherited from mother; NT, not tested. Filled: affected; open: unaffected. Diamonds with 4 represent four individuals, either male or female. All but one individual that are not affected because they carry the mutation on their maternal chromosome 11 and therefore did not develop the disease are marked by an ‘m’. One of the healthy mutation carriers with the mutation on the maternal chromosome has no affected offspring and is not shown in the pedigree for privacy reasons. Also for privacy reasons, five non-affected individuals with the mutation on their paternal chromosome are not indicated. FIG. 11B shows a fluorescence gel image (top panel) and immunoblotting with α-SDHA and α-GAPDH (loading control) antibodies of samples from human tumors, cell lines and mouse tissues. Lanes 1-2, sporadic PGL tumors; lanes 3-5, PGL2 tumors with the hSDH5 G78R mutation; lanes 6-7, cultured human HEK293 and HepG2 cells; lanes 8-9, normal mouse skeletal muscle (skM) and liver tissue. FIG. 11C shows HEK293 cells cotransfected with plasmids expressing wild-type or G78R mutant hSDH5-GFP and Mito-RFP and examined by fluorescence microscopy at 24 h post-transfection. Representative images are shown. FIG. 11D shows HEK293 cells transfected with empty vector (EV) or vectors expressing wild-type (WT) or G78R mutant (MT) hSDH5-Myc. At 30 h post-transfection, cells were harvested and lysates were subjected to immunoprecipitation with α-Myc-conjugated agarose beads. The lysate, eluent and unbound fraction were subjected to SDS-PAGE followed by FAD fluorescence imaging (top panel) and immunoblot with the indicated antibodies (GAPDH, loading control) (lower 3 panels).

### Example 21

### The hSDH5 G78R is a Loss of Function Mutation

FIG. 12A shows wild-type and sdh5Δ mutant strains transformed with empty vector (EV) or plasmids expressing yeast Sdh5-Myc, wild-type human SDH5-Myc or the G78R mutant hSDH5-Myc, all under control of the yeast SDH5 promoter. Serial 5-fold dilutions of saturated SD-Ura liquid culture of each strain were spotted on S-URA solid medium with either 2% glucose or 3% glycerol as the carbon source and grown at 30° C. for 2 days (glucose) or 3 days (glycerol). FIG. 12B shows a fluorescence gel image (top panel) and immunoblotting with the indicated antibodies of whole cell extract from the five strains in FIG. 12A. PGK serves as a loading control. For each strain, FAD fluorescence was normalized to Sdh1 protein level and expressed as a percentage relative to wild-type (bottom panel). Data represent the average±standard deviation of 3 replicates per strain. FIG. 12C shows a model of possible Sdh5 function. Sdh5 is necessary and sufficient for FAD incorporation into Sdh1. Flavo-Sdh1 is then able to stably assemble into the SDH complex to catalyze succinate oxidation and electron transfer.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for screening a test subject to determine risk for developing a succinate dehydrogenase-related disease condition, comprising:
obtaining a biological sample from the test subject; and
identifying a mutation in gene hSDH5 from the biological sample of the test subject, wherein the mutation effectuates a decreased level of succinate dehydrogenase flavination in the test subject as compared to a level of succinate dehydrogenase flavination in a normal subject.

2. The method of claim 1, wherein the mutation is at hSDH5 Gly78 of gene hSDH5.

3. The method of claim 1, wherein the mutation is equivalent to an hSDH5 Gly78Arg substitution.

4. The method of claim 1, wherein the biological sample includes a member selected from the group consisting of biological fluids, biological tissues, biopsies, tumors, cancerous tissue, noncancerous tissue, and combinations thereof.

5. The method of claim 1, wherein the decreased level of succinate dehydrogenase flavination is a substantially complete or complete absence of succinate dehydrogenase flavination.

6. The method of claim 1, wherein the disease condition is a succinate dehydrogenase-related cancer.

7. The method of claim 1, wherein the disease condition includes a member selected from the group consisting of neuroendocrine tumors, paraganglioma tumors, gastrointestinal tumors, Carney-Stratakis syndrome, pheochromocytoma tumors, renal cell carcinomas, optic atrophy, ataxia, myopathies, neurodegeneration, and combinations thereof.

8. The method of claim 1, wherein the disease condition is a paraganglioma tumor.

9. The method of claim 1, wherein results of identifying a mutation in gene hSDH5 are used to affect a member selected from the group consisting of predicting the disease condition risk, predicting the disease condition progression, predicting genetic inheritance risks associated with the disease condition, making a clinical diagnosis of the disease condition, providing information to affect the course of the disease condition, adjusting clinical therapy to treat the disease condition, and combinations thereof.

10. A composition comprising a nucleotide construct of a mutant of hSDH5 and a member selected from the group consisting of a vector, RNA, a virus, and combinations thereof.

11. The composition of claim 10, comprising a vector including the nucleotide construct of mutant hSDH5.

12. The composition of claim 10, wherein the mutant hSDH5 encodes a Gly78 mutation.

13. The composition of claim 12, wherein the mutant hSDH5 encodes a Gly78Arg substitution.

14. A kit for screening a test subject as in claim 1 to determine whether the test subject is at risk for developing a succinate dehydrogenase-related disease condition, comprising:
a kit housing containing an assay capable of identifying a mutation in gene hSDH5 from the biological sample of the test subject;
instructions describing how to use the assay to screen the test subject for the disease condition associated with succinate dehydrogenase flavination.

15. The kit of claim 14, wherein the assay identifies the mutation as being at hSDH5 Gly78 of gene hSDH5.

16. A method for screening a test subject to determine risk for developing a succinate dehydrogenase-related disease condition, comprising:
obtaining a biological sample from the test subject; and
identifying a decreased level of succinate dehydrogenase flavination in the biological sample of the test subject as compared to a level of succinate dehydrogenase flavination in a normal subject.

17. The method of claim 16, wherein the decreased level of succinate dehydrogenase flavination is a substantially complete or complete absence of succinate dehydrogenase flavination.

18. The method of claim 16, wherein the disease condition includes a member selected from the group consisting of neuroendocrine tumors, paraganglioma tumors, gastrointestinal tumors, Carney-Stratakis syndrome, phenochromocytoma tumors, renal cell carcinomas, optic atrophy, ataxia, myopathies, neurodegeneration, and combinations thereof.

19. The method of claim 16, wherein identifying the decreased level of succinate dehydrogenase flavination includes detecting a decrease in SDH1-FAD conjugates in biological sample from the test subject as compared to a level of SDH1-FAD conjugates in a normal subject.

20. The method of claim 16, wherein the decrease in SDH1-FAD conjugates in biological sample from the test subject includes a substantially complete or complete absence of SDH1-FAD conjugates.


SEQUENCE LISTING (24 sequences)

SEQ ID NO: 1 - protein, 166 aa, H. sapiens
MAVSTVFSTSSLMLALSRHSLLSPLLSVTSFRRFYRGDSPTDSQKDMIEIPLPPWQERTDESIETKRARLLYESRKRGMLENCILLSLFAKEHLQHMTEKQLNLYDRLINEPSNDWDIYYWATEAKPAPEIFENEVMALLRDFAKNKNKEQRLRAPDLEYLFEKPR

SEQ ID NO: 2 - protein, 164 aa, M. musculus
MAVVTLIPTLARALSKHSLLSPLPSVTSFRRFYRGDSPTDSQKDMIEIPLPPWQERTDESIETKRARLLYESRKRGMLENCILLSLFAKEYLHNMTEKQLNLYDRLINEPSNDWDIYYWATEAKPAPEIFENEVMELLREFAKNKNKEQRLRAPDLEYLFEKPH

SEQ ID NO: 3 - protein, 158 aa, D. rerio
MFSANIARKVVCSVCRASLRPTVCPVVYRGYRGDAPEPTILEIPLPPWQERAGEALDIKRKRLLYESRKRGMLENCILLSLFAKQYLNTMSESQLKQYDRLINEPSNDWDIYYWATDTQPTPEVYQGEVMDMLKEFTKNRDMEQRLDAPNLEYLDKSS

SEQ ID NO: 4 - protein, 163 aa, D. melanogaster
MLRQLRLTMDISGWIFLPWRRSMSNMKDSPPPPPPLASTFNDVIVDYEDPDYLPLPEYPVRPNEPLETRKQRLLYQSRKRGMLENDLLLSTFAAKHLQNFSAEQTAQYDQLINGVSNDWDIYYWATEVKPTPKEYDTEIMGLLKEHVKNAERVTRLRQPDLNI

SEQ ID NO: 5 - protein, 182 aa, N. crassa
MASSLRALRPAAATFARAAAVRPASVASRHVIAFSRSASNDSTDNQQKKDLDVGELQGATFKIEPLRRVGEDPATMRARLLYQSRKRGTLESDLILSTFAAAHLGSMTPEQLKQYDLFLDENDWDIYYWATQEPPLPGQENQHLQQNSEEASLSEKKEQLPPNPRAGEWAQTVGTFKPAYRP

SEQ ID NO: 6 - protein, 155 aa, C. albicans
MLSKSYRAISKRSFSQSSVVFANYGKPQNPPIDTEIKLKIQPIVRTGETIEVKRARLLYQSRKRGILESDLLLSRFADKYLKTMTMEELDEYDKLLDEADWDIYYWATKNYKITPLPDKWKDSKILKEIQKIANNDEKVIMRMPELNLEPESNTK

SEQ ID NO: 7 - protein, 162 aa, S. cerevisiae
MHNMFPALTKTLSLQGYKIINSQTGSAAWSCGRRWFSSDKDDHDDVVTRIKIAPIKRTNEPLDKKRARLIYQSRKRGILETDLLLSGFAAKYLKKMNEEELEEYDSLLNELDWDIYYWATKNFKTSPLPDKWANSKLLKQLQEFSENKEKEILSMPDLSKYQ

SEQ ID NO: 8 - protein, 139 aa, S. pombe
MLRKTNLSNITTLLRSARCMNRMPQLRFEHTKGDLKRVNRSYETRDAMLARLKYQSRKRGILETDLLLSNFAKDQIDKYPVSLLREYDQLLDEPDWDILYWCSGEREAPEKWKSSQVFKELSKYCRSQRNHTLRMPELF

SEQ ID NO: 9 - protein, 119 aa, C. elegans
MTSRILQRFFTVSTSLRSLTRAEVPGEKIDAKRARLLYQSKKRGILENDILLGDFAEQNLKKMSEPELKAYDKLINGEHMEWDLFYYLSNKKSPPEDVESCQVYQKVKKFVDDKRVPKS

SEQ ID NO: 10 - protein, 101 aa, Sinorhizobium
MTGISRTSADLDPRRRRILFRAWHRGIREMDLILGQFAEAELATLSEAELDELEAIMGEEDNDLVRWITGEKPLPERYATELFFRIAAYRPDFDPVRHETK

SEQ ID NO: 11 - protein, 97 aa, Brucella
MTGSTMATENLDVRRRKLLFRAWHRGMREMDLILGQYADKYIVSFNDDQLNEFEHILEVLDRDLLKWVTGESPSPIPAEYDTPLFRDIVAFRDRVEF

SEQ ID NO: 12 - protein, 90 aa, Rickettsia
MNKLNKNALQKKLFYRSKKRGCREMDYILGSFAEKYLSFMDEKTLKRYTLILDQNDNDLYNWINNKSSAPSYIDSEIIDKLSKIAQIKLL

SEQ ID NO: 13 - DNA, 15 bp, H. sapiens
aagaggggaatgttg

SEQ ID NO: 14 - DNA, 15 bp, H. sapiens
aagaggagaatgttg

SEQ ID NO: 15 - DNA, 17 bp, H. sapiens
accttccggctcagctc

SEQ ID NO: 16 - DNA, 18 bp, H. sapiens
tatcgggcagacgaactc

SEQ ID NO: 17 - DNA, 18 bp, H. sapiens
gttgaccttcccaggctc

SEQ ID NO: 18 - DNA, 20 bp, H. sapiens
gaggttcagctgcttttctg

SEQ ID NO: 19 - DNA, 20 bp, H. sapiens
ggttcagagagactcccagg

SEQ ID NO: 20 - DNA, 20 bp, H. sapiens
gcaacgagagtgaaactcag

SEQ ID NO: 21 - DNA, 21 bp, H. sapiens
ccctggtataggctaacatcg

SEQ ID NO: 22 - DNA, 20 bp, H. sapiens
tgagtacacttgggctgagg

SEQ ID NO: 23 - DNA, 20 bp, H. sapiens
agctctgagcctcaaaagtg

SEQ ID NO: 24 - DNA, 21 bp, H. sapiens
gaagactgtaggaatgagggg